(19) United States
(12) Patent Application Publication
(10) Pub. No.: US 2005/0004646 A1
(43) Pub. Date: Jan. 6, 2005

(54) Energy-activated adhesion layer for drug-polymer coated stent

(76) Inventor: James W. Moriarty JR., Windsor, CA (US)

Correspondence Address: CARDINAL LAW GROUP, Suite 2000, 1603 Orrington Avenue, Evanston, IL, 60201, US

(21) Appl. No.: 10/883,344

(22) Filed: Jul. 1, 2004

Related U.S. Application Data
(60) Provisional application No. 60/484,197, filed on Jul. 1, 2003.

(51) Int. Cl. B05D 3/00; A61F 2/06

(52) U.S. Cl. 623/1.11; 427/2.1; 427/421.1; 427/430.1; 623/1.15

(57) ABSTRACT

The present invention provides a drug-polymer coated stent. The drug-polymer coated stent comprises a stent including a stent framework, an energy-activated adhesion coating disposed on the stent framework, and a drug polymer disposed on the energy-activated adhesion coating. The invention also provides a method of improving adhesion of a polymeric coating on a metallic stent. An adhesion promoter is mixed in a polymeric solution, which is applied to the metallic stent, dried and activated.

### RELATED APPLICATION

This application claims priority to U.S. Provisional Application No. 60/484,197, “Energy-Activated Adhesion Layer for Drug-Polymer Coated Stent” to James W. Moriarty, Jr., filed Jul. 1, 2003, the entirety of which is incorporated by reference.

### FIELD OF THE INVENTION

This invention relates generally to biomedical stents. More specifically, the invention relates to an energy-activated adhesion layer and an improved method of adhesion for a drug-polymer coated metallic stent.

### BACKGROUND OF THE INVENTION

Drug-polymer coated stents typically contain one or more therapeutic compounds within a polymeric matrix disposed on a metallic stent to improve the efficacy of such endovascular stents. These compounds are eluted from the stent coating after the drug-polymer stent is implanted in the body, delivering their patent effects in the tissue bed surrounding the implanted stent. The effectiveness of these drugs is generally improved because of delivery at the point of deployment. The localized levels of the medications may be higher and potentially more effective than orally or intravenously delivered drugs that distribute throughout the body, and may have minimal effect on the impacted area or may be expelled rapidly from the body without reaching their pharmaceutical intent. Drug release from tailored stent coatings may have controlled, timed-release qualities, eluting their bioactive agents over hours, weeks or even months.

For controlled delivery of the pharmaceutical compounds, polymer matrices containing the compounds must be reliably attached to the stent to maintain high quality during manufacturing of such a stent, and to prevent cracking or flaking of the drug-polymer coating when the stent is deployed. The coating may crack or fall off during assembly, packaging, storage, shipping, preparation and sterilization prior to deployment unless effectively adhered to the stent framework. Degradation of the polymer coating may occur with prolonged exposure to light and air, as the constituents of the drug polymer may oxidize or the molecular chains may scission. Although degradation of the polymer coating is of major concern, it is imperative that the adhesion strength of the coating be greater than the cohesive strength of the polymeric matrix to avoid loss of the coating.

Polymeric coatings have a tendency to peel or separate from an underlying metallic stent due to low adhesion strength typical between polymers and metals. Many polymers are non-polar or have limited polarization, reducing their ability to stick to the metal stent framework. Temperature excursions of the coated stent and the difference in thermal expansion coefficients between the metal and the coating may contribute to the fatigue and failure of the bond. Materials that are optimum for drug compatibility and elution may not, in and of themselves, provide sufficient adhesion to a metal substrate. A method to improve the adhesion between a drug-polymer coating and a metallic stent, while retaining the therapeutic characteristics of the drug-polymer stent, would be beneficial. Conventional polymers could be used in the drug-polymer coating. If the adhesive strength of the polymeric coating were improved, a more robust device could be made with lower profiles and the stent struts could touch. Other aspects of the drug-polymer coating, such as cohesive strength, hardness, creep resistance, bulk failure mechanisms and extent of deformation, may impact the robustness of the drug-polymer stent.

It is desirable, therefore, to provide a drug-polymer coated stent with improved adhesion between the drug polymer and the underlying stent framework, to provide a method for improving the adhesion of a polymer coating to a metallic stent, to provide a system for treating heart disease and other vascular conditions using drug-eluting stents with improved adhesion between the drug polymer and the stent, and to overcome the deficiencies and limitations described above.

### SUMMARY OF THE INVENTION

One aspect of the invention provides a drug-polymer coated stent comprising a stent with a stent framework, an energy-activated adhesion coating disposed on the stent framework, and a drug polymer disposed on the energy-activated adhesion coating.

Another aspect of the invention includes a method for improved adhesion of a polymer coating on a metallic stent. A polymeric adhesion promoter is mixed in a solution, after which the adhesion promoter solution is applied to a metallic stent framework and dried. The adhesion coating is activated to improve the adhesion between the polymeric coating and the stent. The solution may include a drug polymer. The method may further include applying a drug-polymer coating to the metallic stent after the adhesion promoter application.

Another aspect of the invention includes a system for treating a vascular condition comprising a catheter, a stent with a stent framework coupled to the catheter, an energy-activated adhesion layer disposed on the stent framework, and a drug-polymer coating disposed on the energy-activated adhesion layer.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a system for treating a vascular condition including a catheter, a stent, a drug-polymer coating, and an energy-activated adhesion layer, in accordance with the current invention;

FIG. 2 is an illustration of a stent cross-section with a drug polymer and an activated adhesion promoter on the stent surface, in accordance with the current invention;

FIG. 3 is an illustration of a stent cross-section with a drug-polymer coating on the stent surface with an energy-activated adhesion coating between the drug-polymer coating and the stent framework, in accordance with the current invention;

FIG. 4 is a flow diagram of one embodiment of a method for manufacturing a drug-polymer coated stent with an activated adhesion promoter, in accordance with the current invention; and

FIG. 5 is a flow diagram of one embodiment of a method for manufacturing a drug-polymer stent with an energy-activated adhesion coating and a drug-polymer coating, in accordance with the current invention.

### DETAILED DESCRIPTION OF THE PRESENTLY PREFERRED EMBODIMENTS

The adhesion of drug-polymer coatings to metallic stents may be improved by the inclusion of polymeric materials that have organic rings and other activatable structures in a polymeric coating disposed on the stent. An activation process may be used to open select organic rings, double bonds, end groups, side chains and molecules of the adhesion promoter. Opening the organic rings, for example, increases the adhesion strength, and thereby improves the adhesion of the coating. An adhesion layer with the adhesion promoter may be applied to the stent and activated, followed by the drug polymer being applied. Alternatively, a drug polymer may be mixed with the adhesion promoter and applied to the stent, and the adhesion promoter then activated for improved adhesion.

One aspect of the present invention is a system for treating coronary heart disease and other vascular conditions, using catheter-deployed endovascular stents with polymeric coatings that include one or more drugs with desired timed-release properties and an adhesion promoter or an adhesion layer. Treatment of vascular conditions may include the prevention or correction of various ailments and deficiencies associated with the cardiovascular system, urinogenital systems, biliary conduits, abdominal passageways and other biological vessels within the body.

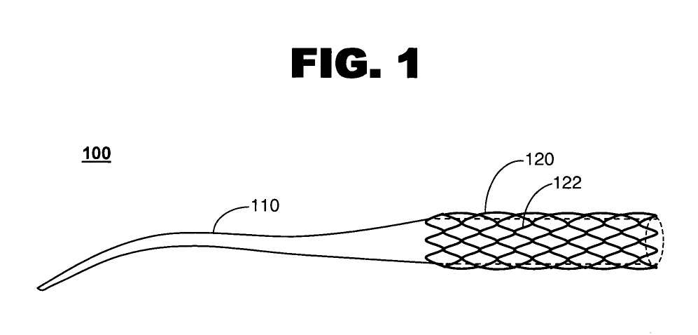

One embodiment of a system for treating a vascular condition, in accordance with the present invention, is illustrated in FIG. 1 at 100. Vascular condition treatment system 100 may include a catheter 110, a stent 120 coupled to the catheter, and a drug-polymer coating 122 with an activated adhesion promoter or an underlying adhesion coating on the stent or stent framework.

The stent is coupled to the catheter, and may be deployed, for example, by pressurizing a balloon coupled to the stent or by retracting a sheath that allows the stent to expand to a prescribed diameter. The stent includes a stent framework. The stent framework may be formed from a base metal such as stainless steel, nitinol, tantalum, MP35N alloy, platinum, titanium, a suitable biocompatible alloy, or other suitable metal alloy.

An adhesion layer may be disposed on the stent framework. The adhesion layer may comprise, for example, maleic anhydride or a polyethylene and maleic anhydride copolymer. Organic rings of the maleic anhydride molecule may be activated and opened, providing additional polarization of the molecule and increasing the bond energy, thus increasing the adhesion to the metallic stent material. The polyethylene or other olefinic material interdispersed with the maleic anhydride therefore would have a higher adhesion energy and improved adhesion to the metallic stent material. Other organic materials that have a ring structure may be used as the adhesion promoter, such that the adhesion promoter is activated when the organic ring structure is opened. Activatable structures such as select organic rings, double bonds, end groups, side chains, and activatable molecules may be activated to improve the adhesion between the adhesion layer or adhesion promoter and the stent framework.

A drug polymer may be disposed on the stent framework. The drug polymer may be mixed with the adhesion promoter and applied, or the drug polymer may be applied to the stent after an adhesion layer is disposed on the stent framework and activated. The adhesion of the drug polymer to a stent coated with a polymeric adhesion layer would be enhanced because the drug polymer would essentially be coating over similar material. The adhesion promoter may be activated prior to or after coating with the drug polymer.

Drug-polymer coating 122 may include one or more drugs. Each drug may include a bioactive agent. The bioactive agent may be a pharmacologically active drug or bioactive compound. The bioactive agent may be eluted from the drug-polymer coating when the stent has been deployed in the body. Alternatively, drug-polymer coating 122 may comprise a bioerodible polymer that erodes after deployment in the body, releasing bioactive agents, drugs and other pharmaceutical agents. Elution refers to the transfer of the bioactive agent out from drug-polymer coating 122. The elution rate is determined by the rate at which the bioactive agent is excreted from drug-polymer coating 122 into the body. The composition of the drug-polymer coating and the interdispersed drugs may control the elution rate of the bioactive agent.

The drug-polymer coating may be subject to degradation during processing, packaging, sterilization, or storage of a drug-polymer coated stent. During sterilization, for example, oxidation of the drug or polymer may occur, resulting in hydrolytic damage, cleavage of the polymeric bonds, breakdown of the polymer and/or drug, or actual cracking or peeling of the drug-polymer coating. Temperature excursions of the in-process or processed stent may incur delamination of all or a portion of the drug-polymer coating. The present invention solves this problem through the use of an adhesion promoter in either the drug polymer or an adhesion layer between the polymer-drug coating and the metallic stent so as to reduce or prevent drug-polymer delamination.

Upon insertion of catheter 110 and stent 120 with drug-polymer coating 122 into a directed vascular region of a human body, stent 120 may be expanded by applying pressure to a suitable balloon inside the stent, or by retracting a sheath to allow expansion of a self-expanding stent. Balloon deployment of stents and self-expanding stents are well known in the art. Catheter 110 may include a balloon used to expand stent 120. Catheter 110 may include a sheath that retracts to allow expansion of the stent.

The adhesion promoter may be interdispersed within drug-polymer coating 122 or within an adhesion coating on the stent, and also may be eluted then metabolized or discarded by the body.

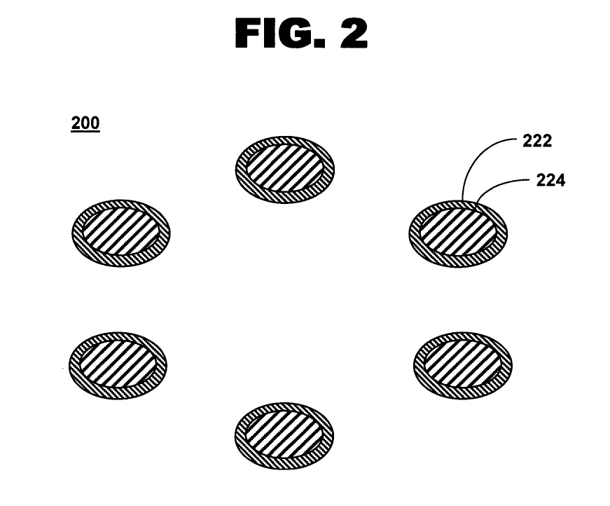

FIG. 2 shows an illustration of a stent cross-section including a drug polymer and an activated adhesion promoter on the stent surface, in accordance with the present invention at 200. Drug-polymer coated stent 200 with an adhesion promoter may include a drug-polymer coating 222 on a stent framework 224. Drug-polymer coating 222 may contain one or more pharmaceutical drugs. Drug-polymer coating 222 may contain a polymeric matrix in which one or more adhesion promoters are interdispersed. One or more adhesion promoters may be interdispersed within drug-polymer coating 222. Drug-polymer coating 222 may contain an energy-activated adhesion layer or coating comprising, for example, an opened organic ring structure, double bond, end group side chain, or activatable molecule.

The drugs and one or more adhesion promoters may be encapsulated in a polymer coating using a microbead, microparticle or nanoencapsulation technology with albumin, liposome, ferritin or other biodegradable proteins and phospholipids, prior to application on the stent.

Stent framework 224 may include a metallic or polymeric base. Stent framework 224 may include a base material of stainless steel, nitinol, tantalum, an MP35N alloy, platinum, or titanium. The stent or stent framework may include a base material of a suitable biocompatible alloy, a suitable biocompatible material including a biodegradable polymeric material, or a combination thereof.

The bioactive agent may include an antineoplastic agent such as triethylene thiophosphoramide, an antiproliferative agent, an antisense agent, an antiplatelet agent, an antithrombogenic agent, an anticoagulant, an antibiotic, an anti-inflammatory agent, a gene therapy agent, an organic drug, a pharmaceutical compound, a recombinant DNA product, a recombinant RNA product, a collagen, a collagenic derivative, a protein, a protein analog, a saccharide, a saccharide derivative, or combinations thereof.

The bioactive agent may be any therapeutic substance that provides a therapeutic characteristic for the prevention and treatment of disease or disorders. An antineoplastic agent may prevent, kill, or block the growth and spread of cancer cells in the vicinity of the stent. An antiproliferative agent may prevent or stop cells from growing. An antisense agent may work at the genetic level to interrupt the process by which disease-causing proteins are produced. An antiplatelet agent may act on blood platelets, inhibiting their function in blood coagulation. An antithrombogenic agent may actively retard blood clot formation. An anticoagulant may delay or prevent blood coagulation with anticoagulant therapy, using compounds such as heparin and coumarins. An antibiotic may kill or inhibit the growth of microorganisms and may be used to combat disease and infection. An anti-inflammatory agent may be used to counteract or reduce inflammation in the vicinity of the stent. A gene therapy agent may be capable of changing the expression of a person's genes to treat, cure or ultimately prevent disease. An organic drug may be any small-molecule therapeutic material. A pharmaceutical compound may be any compound that provides a therapeutic effect. A recombinant DNA product or a recombinant RNA product may include altered DNA or RNA genetic material. Bioactive agents of pharmaceutical value may also include collagen and other proteins, saccharides, and their derivatives.

For example, the bioactive agent may be selected to inhibit vascular restenosis, a condition corresponding to a narrowing or constriction of the diameter of the bodily lumen where the stent is placed. The bioactive agent may generally control cellular proliferation. The control of cell proliferation may include enhancing or inhibiting the growth of targeted cells or cell types.

The bioactive agent may be an agent against one or more conditions including coronary restenosis, cardiovascular restenosis, angiographic restenosis, arteriosclerosis, hyperplasia, and other diseases and conditions. For example, the bioactive agent may be selected to inhibit or prevent vascular restenosis, a condition corresponding to a narrowing or constriction of the diameter of the bodily lumen where the stent is placed. The bioactive agent may generally control cellular proliferation. The control of cell proliferation may include enhancing or inhibiting the growth of targeted cells or cell types.

The bioactive agent may include podophyllotoxin, etoposide, camptothecin, a camptothecin analog, mitoxantrone, rapamycin, and their derivatives or analogs. Podophyllotoxin is an organic, highly toxic drug that has antitumor properties and may inhibit DNA synthesis. Etoposide is an antineoplastic that may be derived from a semi-synthetic form of podophyllotoxin to treat monocystic leukemia, lymphoma, small-cell lung cancer, and testicular cancer. Camptothecin is an anticancer drug that may function as a topoisomerase inhibitor. Related in structure to camptothecin, a camptothecin analog such as aminocamptothecin may be used as an anticancer drug. Mitoxantrone is also an important anticancer drug, used to treat leukemia, lymphoma, and breast cancer. Rapamycin or sirolimus is a medication that may interfere with the normal cell growth cycle and may be used to reduce restenosis. The bioactive agent may also include analogs and derivatives of these agents. Antioxidants may be beneficial on their own rights for their antirestonetic properties and therapeutic effects.

Drug-polymer coating 222 may soften, dissolve or erode from drug-polymer coated stent 200 to elute at least one bioactive agent. This elution mechanism may be referred to as surface erosion where the outside surface of the drug-polymer coating dissolves, degrades, or is absorbed by the body; or bulk erosion where the bulk of the drug-polymer coating biodegrades to release the bioactive agent. Eroded portions of the drug-polymer coating may be absorbed by the body, metabolized, or otherwise expelled.

The pharmaceutical drug may separate within drug-polymer coating 222 and elute the bioactive agent. Alternatively, the pharmaceutical drug may erode from drug-polymer coated stent 200 and then separate into the bioactive agent. The pharmaceutical drug or agent may be eluted and absorbed or expelled by the body. Drug-polymer coating 222 may include multiple pharmaceutical drugs, and more than one adhesion promoter. Drug-polymer coating 222 may include a single bioactive agent with various adhesion promoters to secure the bioactive agent to the stent framework. Drug-polymer coating 222 may comprise an energy-activated adhesion promoter disposed on the stent framework. The energy-activated adhesion promoter may comprise, for example, a polyethylene and maleic anhydride copolymer, maleic anhydride, an adhesion promoter with an organic ring structure, or any suitable polymeric adhesion promoter.

Drug-polymer coating 222 may also include a polymeric matrix. For example, the polymeric matrix may include a caprolactone-based polymer or copolymer, or various cyclic polymers. The polymeric matrix may include various synthetic and non-synthetic or naturally occurring macromolecules and their derivatives. The polymeric matrix may include biodegradable polymers such as polylactide (PLA), polyglycolic acd (PGA) polymer, poly (e-caprolactone) (PCL), polyacrylates, polymethacryates, or other copolymers. The pharmaceutical drug may be dispersed throughout the polymeric matrix. The pharmaceutical drug or the bioactive agent may diffuse out from the polymeric matrix to elute the bioactive agent. The pharmaceutical drug may diffuse out from the polymeric matrix and into the biomaterial surrounding the stent. The bioactive agent may separate from within drug-polymer coating 222 and diffuse out from the polymeric matrix into the surrounding biomaterial.

The polymeric matrix may be selected to provide a desired elution rate of the bioactive agent. The pharmaceutical drugs may be synthesized such that a particular bioactive agent may have two different elution rates. A bioactive agent with two different elution rates, for example, would allow rapid delivery of the pharmacologically active drug within twenty-four hours of surgery, with a slower, steady delivery of the drug, for example, over the next two to six months. The adhesion promoters may be selected to firmly secure the rapidly deployed bioactive agents and the slowly eluting pharmaceutical drugs to the stent framework.

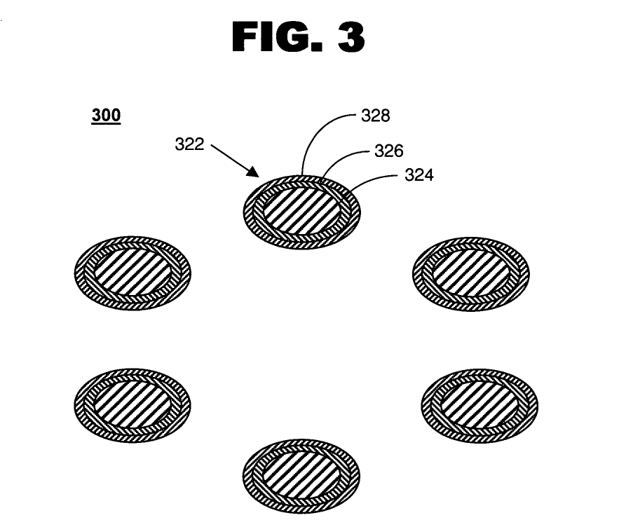

FIG. 3 shows an illustration of a stent cross-section comprising a polymeric coating containing a drug-polymer coating disposed on an adhesion coating between the drug-polymer coating and the stent framework, in accordance with another embodiment of the present invention at 300. Drug-polymer coated stent 300 with polymeric coating 322 includes an adhesion layer 326 on a stent framework 324 and a drug-polymer coating 328 on adhesion layer 326. Adhesion layer 326 may be referred to herein as an adhesive coating. Drug-polymer coating 328 includes at least one interdispersed bioactive agent. Adhesion layer 326 may be void or nearly void of pharmaceutical drugs.

Adhesion layer 326 may be selected to improve the adhesion and minimizing the likelihood of delamination of the polymeric coating from stent framework 324. Metal-adhering attributes of adhesion promoters in the adhesion layer aid in the adhesion between the polymeric coating and the metallic stents.

Adhesion layer 326 may be comprised of any suitable adhesion material that enhances adhesion between drug-polymer coating 328 and stent framework 324. The energy-activated adhesion coating or adhesion layer 326 may comprise an opened organic ring structure. One suitable adhesion material is a maleic anhydride and polyethylene copolymer. Other suitable adhesion materials include polymers with organic rings, double bonds, end groups, side chains and activatable molecules that can be opened to increase the bond energy and adhesive strength of the adhesion layer.

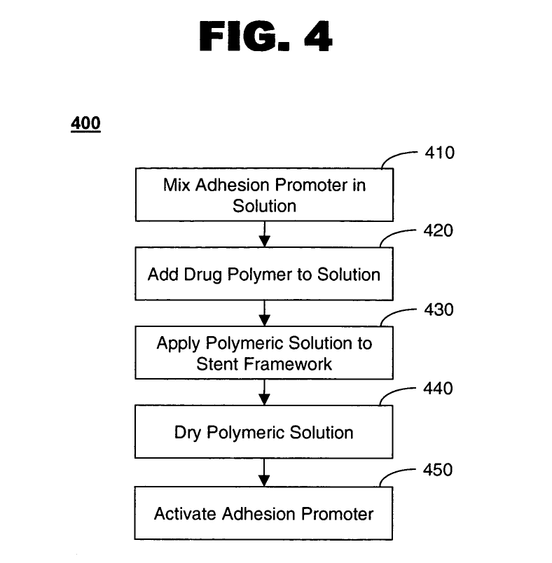

Another aspect of the current invention is a method of manufacturing a drug-polymer coated stent with an adhesion promoter. FIG. 4 shows a flow diagram of one embodiment of a method for manufacturing a drug-polymer coated stent including an activated adhesion promoter, in accordance with the present invention at 400.

The drug-polymer coated stent with an activated adhesion promoter is manufactured by mixing a polymeric adhesion promoter in a polymeric solution, as seen at block 410. Water, an alcohol such as methanol or ethanol, or other suitable solvents may be used to form the solution. The polymeric adhesion promoter may include a polymer with an organic ring structure that can be activated. The polymeric adhesion promoter may include maleic anhydride. The polymeric adhesion promoter may include a polyethylene and maleic anhydride copolymer. The polymeric solution may comprise a drug polymer.

A drug polymer may be added to the polymeric solution, as seen at block 420. The drug polymer may include a polymeric matrix and one or more therapeutic compounds. Alternatively, the drug polymer may be mixed with a suitable solvent to form a polymeric solution, and the polymeric adhesion promoter is then added to the polymeric solution.

To form a drug-polymer coating, a polymer such as a vinyl acetate derivative may be mixed with other polymers or monomers in a solvent such as isopropyl alcohol and added to the polymeric solution. The mixture may be reacted to form new polymers or modify existing polymers in the polymeric solution. One or more bioactive agents may be mixed with the polymerized mixture to form a drug polymer with a predefined elution rate. A suitable bioactive agent or a solution containing the bioactive agent may be mixed in with the polymeric solution up to 75 percent bioactive agent or greater by weight in the drug-polymer coating. Alternatively, a polymer such as a copolyester or block copolymer may be dissolved in a suitable solvent, and one or more bioactive agents may be added to the mixture. The mixture may be combined with the adhesion promoter in the polymeric solution. One or more adhesion promoters may be selected and added to the mixture.

The polymeric solution is applied to stent framework, as seen at block 430. The polymeric solution may be applied to the stent by dipping, spraying, painting, brushing or any other suitable method for applying the polymer solution.

Excess liquid may be blown off and the film dried, as seen at block 440. Drying of the polymeric solution to eliminate or remove any volatile components may be done at room temperature or elevated temperatures under dry nitrogen or other suitable environment. A second dipping and drying step may be used to thicken the coating. The thickness of the drug-polymer coating may range between 1.0 microns and 200 microns or greater in order to provide satisfactory pharmacological benefit with the bioactive agent. This action may provide a coating on the stent, although it may not provide sufficient energy to open the organic ring structure, double bond, end group, side chain, or activatable molecule and thus provide effective bond sites to the metal substrate.

The adhesion promoter may be activated after the drying step, as seen at block 450. The polymer in contact with the metal may be supplied activation energy to activate the adhesion promoter. The adhesion promoter may be activated, for example, when an organic ring structure, double bond, end group, side chain, or activatable molecule of the adhesion promoter is opened. Activation energy may be supplied to the chemical structure of the adhesion promoter to provide a source of bonding to the metallic base of the stent. The adhesion promoter may be activated by heating the coated stent to an elevated temperature. Alternatively, the coated stent may be heated to nearly the melting temperature of the polymeric coating, or to at least a suitable temperature for breaking at least one of the organic rings in the adhesion promoter to increase the adhesive strength. The coated stent may be irradiated with laser radiation at one or more prescribed wavelengths to selectively break one of the bonds in the adhesion promoter. The adhesion promoter may be activated, for example, by exposing the coated stent to an x-ray irradiation source.

The adhesion promoter may be activated by heating the metallic stent framework. The polymer in contact with the metal may be supplied activation energy to form a bond on the stent. Induction heating, for example, will heat the stent metal, which in turn will heat the adhesion promoter in contact with the metal, thereby breaking the organic rings and increasing the adhesive strength of the polymeric adhesion promoter. Other methods of heating the metallic stent or of energy-activating the adhesive coating may be employed, such as laser irradiation, x-ray irradiation, or any other suitable method for activating the adhesion promoter. The adhesion promoter may be activated using an activation system such as an induction heater, an oven, a laser irradiation system, an x-ray irradiation system, or any other suitable activation system.

The coated stent may be integrated into a system for treating vascular conditions such as heart disease by assembling the coated stent onto a catheter. Finished coated stents may be reduced in diameter and placed into the distal end of the catheter, formed with an interference fit that secures the stent onto the catheter. The catheter with the stent may be placed in a catheter package and sterilized prior to shipping and storing. Sterilization using conventional medical means occurs before clinical use.

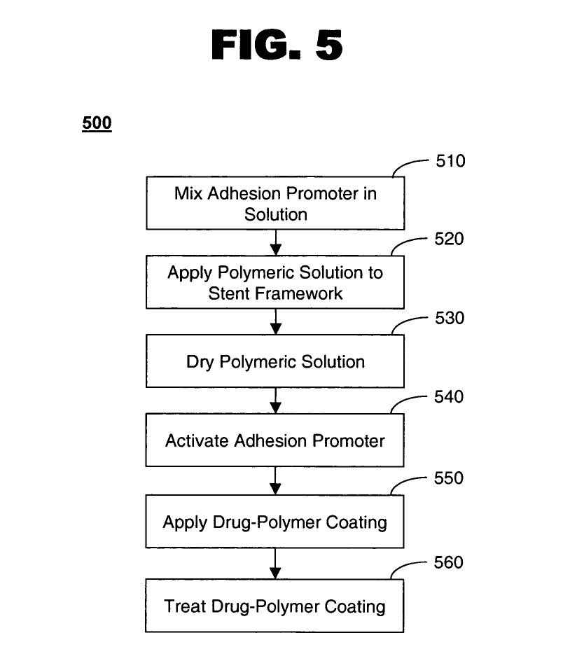

Another aspect of the current invention is a method of manufacturing a drug-polymer coated stent with a drug-polymer coating disposed on top of an adhesion layer. FIG. 5 shows a flow diagram of one embodiment of a method for manufacturing a drug-polymer coated stent with an energy-activitated adhesion coating and a drug-polymer coating, in accordance with the current invention at 500.

The drug-polymer coated stent may be manufactured by mixing a polymeric adhesion promoter in a solution, as seen at block 510. An alcohol such as methanol or ethanol, water, or other suitable solvents may be used to form the polymeric solution. The polymeric adhesion promoter may include a polymer with an organic ring structure that can be activated, such as maleic anhydride. The polymeric adhesion promoter may include, for example, a polyethylene and maleic anhydride copolymer.

The polymeric solution is applied to a metallic stent or a metallic stent framework, as seen at block 520. The polymeric solution may be applied to the stent framework by dipping, spraying, painting, brushing, or by other suitable methods. The polymeric solution disposed on the metallic stent framework is dried, as seen at block 530. Excess liquid may be blown off and the film dried to form an adhesion coating. Additional application and drying steps may be included to reach the desired thickness of the adhesion coating.

The adhesion promoter may be activated after the drying step, as seen at block 540. The polymer in contact with the metal may be supplied with sufficient activation energy. The adhesion promoter may be activated, for example, when an organic ring structure, double bond, end group, side chain or activatable molecule of the adhesion promoter is opened. The adhesion promoter on the coated stent or the metallic stent framework is activated by using any suitable energy-activation system such as an induction heater, an oven, a laser irradiation system, or an x-ray irradiation system. The coated stent may be heated to a prescribed temperature, for example, for breaking or opening at least one of the organic rings in the adhesion promoter to increase the adhesive strength. In another example, the coated stent may be irradiated with a laser operating at a suitable wavelength for breaking select bonds or organic rings in the adhesion promoter.

A drug-polymer coating is applied to the energy-activated adhesion layer comprising the activated adhesion promoter, as seen at block 550. The drug polymer may be mixed in a suitable solvent, and applied to the adhesion layer using an application technique such as dipping, spraying, painting or brushing. In subsequent coating operations, for example, a drug-polymer coating with olefinic polymers may adhere well over an adhesion layer comprising olefinic polymers.

The drug-polymer coating may be treated, as seen at block 560. Treatment of the drug-polymer coating may include air drying or low-temperature heating in air, nitrogen, or other controlled environment. The drug-polymer coating may be treated by heating the drug-polymer coating to a predetermined temperature.

The coated stent with the drug-polymer coating disposed on the energy-activated adhesion layer may be coupled to a catheter.

Although the present invention applies to cardiovascular and endovascular stents with timed-release pharmaceutical drugs, the use of adhesion promoters in polymer-drug coatings and adhesion layers may be applied to other implantable and blood-contacting biomedical devices such as coated pacemaker leads, microdelivery pumps, feeding and delivery catheters, heart valves, artificial livers and other artificial organs.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A drug-polymer coated stent, comprising:
a stent including a stent framework;
an energy-activated adhesion coating disposed on the stent framework; and
a drug polymer disposed on the energy-activated adhesion coating.

2. The drug-polymer coated stent of claim 1, wherein the stent framework comprises a base metal selected from the group consisting of stainless steel, nitinol, tantalum, MP35N alloy, platinum, titanium, a biocompatible alloy, and a metal alloy.

3. The drug-polymer coated stent of claim 1 wherein the energy-activated adhesion coating comprises an opened organic ring structure.

4. The drug-polymer coated stent of claim 1 wherein the energy-activated adhesion coating comprises a polyethylene and maleic anhydride copolymer.

5. A method of improving adhesion of a polymeric coating on a metallic stent, comprising:
mixing an adhesion promoter in a polymeric solution;
applying the polymeric solution to a metallic stent framework;
drying the polymeric solution disposed on the metallic stent framework; and
activating the adhesion promoter.

6. The method of claim 5 wherein the adhesion promoter comprises an organic ring structure.

7. The method of claim 5 wherein the adhesion promoter comprises maleic anhydride.

8. The method of claim 5 wherein the adhesion promoter comprises a polyethylene and maleic anhydride copolymer.

9. The method of claim 5 wherein the polymeric solution comprises a drug polymer.

10. The method of claim 5 wherein the polymeric solution is applied using an application technique selected from the group consisting of dipping, spraying, painting and brushing.

11. The method of claim 5 wherein the adhesion promoter is activated when an organic ring structure of the adhesion promoter is opened.

12. The method of claim 5 wherein the adhesion promoter is activated using an activation system selected from the group consisting of an induction heater, an oven, a laser irradiation system, and an x-ray irradiation system.

13. The method of claim 5 further comprising;
applying a drug-polymer coating to the activated adhesion promoter disposed on the metallic stent framework; and
treating the drug-polymer coating.

14. The method of claim 13 wherein the drug-polymer coating is applied using an application technique selected from the group consisting of dipping, spraying, painting and brushing.

15. The method of claim 13 wherein the drug-polymer coating is treated by heating the drug-polymer coating to a predetermined temperature.

16. A system for treating a vascular condition, comprising:
a catheter;
a stent coupled to the catheter, the stent including a stent framework;
an energy-activated adhesion layer disposed on the stent framework; and
a drug-polymer coating disposed on the energy-activated adhesion layer.

17. The system of claim 16 wherein the stent framework comprises a base metal selected from the group consisting of stainless steel, nitinol, tantalum, MP35N alloy, platinum, titanium, a biocompatible alloy, and a metal alloy.

18. The system of claim 16 wherein the energy-activated adhesion layer comprises an opened organic ring structure.

19. The system of claim 16 wherein the energy-activated adhesion layer comprises maleic anhydride.

20. The system of 16 wherein the energy-activated adhesion layer comprises a polyethylene and maleic anhydride copolymer.
